(19) United States
(12) Patent Application Publication
(10) Pub. No.: US 2006/0241432 A1
(43) Pub. Date: Oct. 26, 2006

(54) Method and apparatus for calibration, tracking and volume construction data for use in image-guided procedures

(75) Inventors: Alan J. Herline, Nashville, TN (US); John R. Warmath, Nashville, TN (US); Philip Bao, Nashville, TN (US); Robert L. Galloway, Nashville, TN (US)

Correspondence Address: AKIN GUMP STRAUSS HAUER & FELD L.L.P., ONE COMMERCE SQUARE, 2005 MARKET STREET, SUITE 2200, PHILADELPHIA, PA, 19103, US

(73) Assignee: Vanderbilt University

(21) Appl. No.: 11/353,576

(22) Filed: Feb. 14, 2006

Related U.S. Application Data
(60) Provisional application No. 60/652,953, filed on Feb. 15, 2005.

(51) Int. Cl. A61B 8/00

(52) U.S. Cl. 600/437

(57) ABSTRACT

An apparatus that collects and processes physical space data while performing an image-guided procedure on an anatomical area of interest includes a calibration probe that collects physical space data by probing a plurality of physical points, a tracked ultrasonic probe, a tracking device that tracks the ultrasonic probe in space and an image data processor. The physical space data provides three-dimensional coordinates for each of the physical points. The image data processor includes a computer-readable medium holding computer-executable instructions. The executable instructions include determining registrations used to indicate position in both image space and physical space based on the physical space data collected by the calibration probe; using the registrations to map into image space, image data describing the physical space of the tracked ultrasonic probe used to perform the image-guided procedure and the anatomical area of interest; and constructing a three-dimensional volume based on the ultrasonic image data on a periodic basis.

### CROSS REFERENCE TO RELATED APPLICATIONS

This application claims priority to U.S. Provisional Patent Application No. 60/652,953 filed on Feb. 15, 2005 entitled “Method and Apparatus for Calibration, Tracking and Volume Construction Data for Use in Image-Guided Procedures.”

### BACKGROUND OF THE INVENTION

The present invention relates to an apparatus and method for calibration, tracking and volume construction data for use in image-guided procedures, and more particularly, to an apparatus and method for calibration, tracking and volume construction data for use in image-guided procedures within the context of anatomical imaging.

The majority of the background work was performed using an Endorectal ultrasound probe. However, this work is also applicable in other anatomical areas of interest using both flexible and rigid ultrasound probes. These areas include the colon, esophagus, pancreas, duodenum, liver, breast, kidney, heart, prostate, and any other part of the body that can be imaged by ultrasound. The following detailed description describes embodiments of the present invention as used for the rectum.

Rectal cancer is a growing problem in the world today, and it has been estimated that 42,000 new cases will be diagnosed in the United States in 2003. The annual rate has been growing and the percentage of cases in which rectal cancer is determined to be the primary cause of death has increased. In 1999 there were only 34,700 estimated cases, but in approximately 20% of these cases cancer was determined to be the primary cause of death for the patient. It is expected that approximately 7,000 more cases will be diagnosed in 2003 as compared to 1999, with this number growing at a rate of approximately 1000-3000 cases per year.

Rectal cancer generally comprises cancer cells found living in the tissues of the rectum. Most rectal cancers are sporadic; even hereditary diseases such as familial adenomatous polyposis (FAP), begin with a spontaneous mutation in the tumor suppressor gene, adenomatous polyposis coli (APC) that causes it to become inactivated. Since the APC gene helps to regulate the growth of new cells (i.e., it is a tumor suppressor gene), the inactivation of it disrupts the natural balance found in healthy tissue and allows the over production of potentially malignant tissue. This inactivation also causes the hypomethylation of DNA, which may lead to k-ras activation. K-ras is an oncogene which, when permanently activated, causes uncontrollable growth of cells. If the activation of K-ras is coupled with the inactivation of the APC gene, the opportunity for the uncontrollable growth of new tissue is even greater.

Most new colorectal growths, or neoplasms, do not begin as malignant tumors. Most new growths in the rectum begin as adenomas or adenomatous polyps. An adenoma is a benign neoplasm of glandular epithelium. There are two types of glandular features by which adenomas are classified, namely, villous and tubular. Villous adenomas are flat and found in the rectum more often than tubular; they also are at increased risk of becoming malignant. Tubular adenomas tend to be on a stalk and found in the colon or rectum. The malignant tissues formed from adenomas are called carcinomas, giving rise to the name adenocarcinoma (a malignant tumor arising from an adenoma). In rectal cancer, most adenocarcinomas originate from polyps, but not all polyps found in the rectum are adenomas. The rate at which polyps transform to cancer is related to size and type (tubular vs. villous) and is reported to be between about 2 and 9.3 percent. Because of the risk of malignant transformation, polyp removal prior to cancerous transformation is the best clinical option.

Hereditary nonpolyposis colorectal cancer (HNPCC) is an inherited disease of importance that causes rectal cancer. HNPCC is caused by a mutation in one of the genes that codes for DNA repair. The following is a list of the genes can be altered causing HNPCC: hMSH2, hMLH1, hPMS1 and HPMS2. When this mutated gene does not reconstruct the DNA in the correct sequence, errors occur that can lead to cancerous growth. This disease presents itself at an earlier age, and the tumors tend to be villous. The Amsterdam Criteria was defined in 1991 for research purposes and is a way to classify a tumors as HPNCC. Surgeons continue to apply the criteria to clinical cases, and therefore, it has become important in colorectal cancer diagnosis. The criteria tell physicians to consider HNPCC when (i) three family members have colon cancer, (ii) two generations of the family have colon cancer, and (iii) at least one individual was diagnosed with the disease before the age of 50. However, these criteria do not work well in a population-based examination for colon cancer because up to 2% of non-hereditary colon cancer patients meet them. There are also other types of malignant rectal tumors, including endocrine tumors (carcinoids) and Kaposi sarcomas, that will not be the focus of this thesis.

Besides being an inheritable disease, many studies have shown that dietary factors can influence the onset of sporadic rectal cancer. It is a common belief that there are also other risk factors such as (i) a large, daily caloric intake, (ii) a large percentage of dietary fat, (iii) being obese, (iv) an inadequate fiber intake, and (v) a low percentage of fresh fruits and vegetables. There have not been enough conclusive epidemiological studies to determine a percent intake of dietary fat that causes cancer or percent intake of fiber that might be protective. It is also not known how the combination of a high fat/low fiber diet compares in the rate of rectal cancer occurrence to the combinations of high fat/high fiber or low fat/low fiber diets. Whatever the future research may show, there are a few conclusions that can be drawn from the work that has already done and the observations that have already been made. The most obvious conclusion is that especially for those people in high-risk families for rectal cancer, a controlled diet that is low in fat and high in fiber, fruits, and vegetables has a chance of decreasing the probability of developing polyps and hopefully rectal cancer.

The rectum begins at the lower end of the colon when the longitudinal bands of muscle (Taenia) surrounding the colon coalesce. It continues for approximately 15 cm until it narrows and forms the anus. The transition region from the rectum to the anus is accompanied by the bowel narrowing as it passes through the levator muscles. In this region, the composition of the rectal wall changes making tumor staging difficult. “The rectum is arbitrarily divided into three portions, the lower rectum (0-6 centimeters (cm)), the middle rectum (7-11 cm) and the upper rectum (12-15 cm). These categories are useful in planning surgical approach. The lower rectum is normally found to be dilated when compared to the middle and upper sections. Also, there are differences in the lymphatic systems draining the lower and middle/upper rectum. Lymphatic drainage from the upper rectum is exclusively upwards along the superior rectal vessels. While lymph drainage from the lower rectum may take any of three routes, the main direction of spread from tumors in any part of the rectum is upwards. The differences in lymphatic drainage can also cause differences in surgical approaches among the three regions. However, the main anatomical difference in the three regions that affects the surgical approach is the pelvis. Because of the bony pelvis, the principle of wide local removal of the cancer-bearing bowel segment is subject to severe limitations by the anatomy of the pelvic rectum.

To understand the surgical treatment of a rectal tumor, it is important to first understand the anatomy of the rectum. A representation of the layers of the rectum is shown in FIG. 2. The rectal wall is made up of six layers, and the amount of growth of a tumor into these layers helps to classify it into its proper stage. The six layers from the lumen outward are: 1) mucosa, 2) muscularis mucosa, 3) submucosa, 4) muscularis propria, 5) subserosal connective tissue (subserosa), and 6) serosa. The layers that are considered important in the TNM (tumor, node, metastasis) system for classifying tumors are the submucosa, muscularis propria, and the subserosa. The mucosal layers are hyperechoic, and the muscular layers are hypoechoic thereby making it feasible to use endorectal ultrasound in T staging. The image shown in FIG. 3 was taken with an ERUS probe and clearly shows these layers as would be seen clinically in a rectum with no cancer.

Staging was historically performed clinically and evaluated post-operatively using the pathology specimen. In the early 1980's clinicians realized the importance of ERUS in rectal cancer staging, and in 1985 the accepted staging methods were revised to include ERUS. The layers of the rectal wall with a representation of degree of invading tumors (stage) are shown in FIG. 4, and the standard TNM staging definitions for primary tumors, regional lymph nodes, and distant metastases are shown in Table 1.

TABLE 1
T Staging Definitions for Rectal Cancer
From the American Joint Committee on
Cancer's 5^{th} edition Cancer Staging manual,

| Stage | Definition |
|---|---|
| Primary tumor (T) | |
| TX | Primary tumor cannot |
| | be assessed |
| T0 | No evidence of primary |
| | tumor |
| Tis | Carcinoma in situ: |
| | intraepithelial or |
| | invasion of lamina |
| | propria |
| T1 | Tumor invades |
| | submucosa |
| T2 | Tumor invades |
| | muscularis propria |
| T3 | Tumor invades through |
| | muscularis propria into |
| | the subserosa or into |
| | nonperitonealized |
| | pericolic or perirectal |
| | tissues |
| T4 | Tumor perforates |
| | visceral peritoneum or |
| | directly invades other |
| | organs or structures |

Currently, there is not an accurate manor to stage lymph nodes associated with rectal cancer. CT and MRI both have reported accuracies between 60 and 65 percent while ERUS has reported accuracies ranging from 58 to 81 percent. Improved N-staging techniques should improve benign and malignant lymph node detection in the future. However, a malignant lymph node does not mean that there is spread of the cancer to other sites, and metastatic spread can occur without involved lymph nodes. Therefore, improved lymph node staging does not assure an improvement in the outcome of rectal cancer. While this technology may improve M staging, this thesis is focused on the T staging of rectal cancer with endorectal ultrasound.

There are many different techniques that researchers and clinicians have tried to use to detect and stage rectal cancer. Some commonly used screening techniques include the Fecal occult blood test (FOBT), the Digital Rectal Exam (DRE), Sigmoidoscopy and Colonoscopy. Some commonly used staging techniques include DRE, Positron Emission Tomography (PET), Magnetic Resonance Imaging (MRI), Computed Tomography (CT), Endorectal Ultrasound (ERUS) and Trans-vaginal Ultrasound (TVUS). Reliable screening and staging techniques are important parts of the prevention and treatment of rectal cancer. However, improvements are needed in both screening and staging.

Attempts have been made to use PET for T staging of rectal cancer, but have not been successful. The PET technique provides a functional image with limited anatomical detail. Therefore, it is thought that it is useful in looking at the recurrence of the cancer, or at the spread of metastatic cells to other organs, but is currently not useful in the anatomic, primary staging of rectal cancer. However, it has been proposed that the combination of PET with either CT or MR for this purpose may be of use in the future

MRI has seen limited clinical use for various reasons, one of which being that there has not been an overwhelming consensus of its utility by the surgeons treating rectal cancer. The possibility of using MRI for the preoperative staging of rectal cancer was introduced in 1986 and was determined to be good for M staging. However, with only a few exceptions, the consensus of the majority of the experts in the field show that there are major limitations for a widespread use of MR in T staging of rectal cancer using body coils. One major disadvantage is that it is likely to overstage T2 tumors. However, MRI has shown been useful in determining the difference in mucinous and nonmucinous rectal carcinomas, and MR has also been used to look at the local recurrence of rectal cancer or the spread of the cancer to other organs in the body may be of clinical use. Even so, the use of MR in these situations is not wide spread in the United States.

With the introduction of endorectal coils, the accuracies of T staging with MRI has greatly improved, rivaling that of ERUS. One study reported accuracies of 92% for T1-T2 tumors and 94% in T3 tumors as compared to post-operative pathological results. These coils, combined with different pulse sequences, have allowed for the visualization of the individual layers of the rectal wall. However, this same study reported some problems with this system that limits its usefulness as a T staging technique. This procedure requires a long examination time, the costs are relatively high, and movement-related artifacts in the images sometimes decreases the resolution so that the individual layers of the rectum cannot be seen. Another study used an endorectal coil MRI and found it to be a reliable local staging technique for rectal cancer, but even though its accuracy is very high, ERUS is still the preferred method because of ease of application and cost.

With the continued efforts to reduce the cost of MRI, the improvements in the resolution of higher field MR magnets, and the new applications that may potentially be discovered, the practicality of its use either in T staging or looking at rectal cancer recurrence may increase in the future. A group in Austria and Germany has just reported the possibility of using a value, called the perfusion index of a tumor, to predict the outcome of adjuvant preoperative therapy and to introduce MR as a potential screening technique. The perfusion index is calculated from a preoperative, dynamic, T1-weighted image set taken of a primary rectal carcinoma. This type of noninvasive, predictive screening is one of the many new ideas that could prove to be of great value in the future.

Computed tomography (CT) is another imaging modality that, like MRI, provides tomographic slices of the imaged volume. When CT was first introduced into the role of T and M staging, it was thought to be very accurate. However, for T staging, its many limitations were quickly discovered. It does not differentiate the layers of the rectal wall. CT has a wide spread of inter- and intra-observer accuracies, and is inaccurate in the identification of lymph node metastases. The ability to detect local tumor extension is stage dependent, being accurate in the identification of late stage (T4) tumors, and very poor at the identification and differentiation of T2 and T3 tumors.

CT has been utilized in three other areas including the selection of patients for pre-operative adjuvant therapy, the tracking of recurrent cancer postoperatively and looking at systemic disease (i.e., liver metastases). Because CT provides an anatomic image, it allows a view of the tumor in relation to the surrounding structures. It also shows liver metastases (M staging), if the cancer has spread metastatically. Therefore CT is a good choice for use in the selection of patients for pre-operative therapy. However, using CT to look for recurrent tumors has problems. In CT, a recurrent tumor tends to image like a local soft tissue mass, typically with central hypodensities, after intravenous contrast injection. But, a major difficulty with CT in detecting recurrent cancer is its insensitivity to local tumor at the anastomatic site, inability to detect tumor within a fibrotic surgical scar, and inability to differentiate between hyperplastic and tumorous lymph nodes. While ERUS is the preferred method to detect early local recurrence, no single method is outstanding. PET can be useful for tumors greater than about 0.5 cm and that actively uptake FDG, and even with the problems that exist using CT, it is worth noting again that it is a good technique to use in the identification of late stage tumors and M staging, as well as in the diagnostic work-up of patients for pre-operative adjuvant therapy. Therefore, CT used in combination with other imaging modalities, provides useful information for the staging and treatment of rectal cancer.

Endorectal ultrasound has emerged as the imaging modality of choice for staging rectal cancer and has been shown to be especially accurate for early stage tumors (T1,T2) and tumors that penetrate the rectal wall (T3). As shown in FIG. 5, an ERUS image clearly shows a rectal tumor along with the deformed layers of the rectal wall.

Some of the problems using ERUS arise from 1) inter-user variability (including T2 vs T3 discrepancies), 2) user to user portability, 3) adjuvant therapy, and 4) stenosis. Inter-user variability and the wide range in reported staging accuracies are due to the inflammation of some tumors as well as the non-tomographic nature of ERUS. Another problem encountered with the use of ERUS is that adjuvant, preoperative radiotherapy can increase the echogenicity of the rectal wall thereby reducing its accuracy in T staging. ERUS is also less accurate in the lower rectum because changes in anatomy cause the examination to be more difficult; although, the position of the tumor with respect to the circumference of the rectum does not decrease the accuracy in T staging. There have been problems with overstaging and understaging of mid-stage tumors for two reasons: 1) the overstaging is caused by the lack of differentiation between tumor and inflammation and is a problem associated with T2 tumors, and 2) the understaging is thought to be caused by a lack of specific, cellular information at the microscopic level. With ultrasound, the resolution of the image can be improved by increasing the frequency. However, there is a trade off for increased frequency, which is decreased imaging depth. Therefore a useful balance of imaging resolution and depth must be found. Frequencies from 5 to 10 MHz have been shown to provide acceptable results.

ERUS is limited in the visualization of stenotic tumors of the rectum. Approximately 17 percent of stenotic tumors are impossible to stage with ERUS. In women this problem is currently being solved with the use of transvaginal ultrasound (TVUS) because of the improved visualization with this technique. However, this problem will not be solved in men unless improvements are made in the ERUS systems, or another imaging modality is shown to image stenotic tumors well.

Despite the many limitation, ERUS is a very valuable technique used in the staging of rectal cancer. It is inexpensive, relatively easy to use, and identifies early stage and T3 tumors extremely well. It is important for the clinician to understand its limitations and also its potential benefits when combined with other forms of imaging. The combination of DRE, ERUS, and CT seems to be the most accurate, most commonly used, and most cost effective for the complete staging and operative planning of rectal cancer

Endorectal ultrasound is the current standard of care for T staging of rectal cancer. However, ERUS in its current form is not readily portable among examiners. Some common sources of error when using ERUS include false instrumentation, interpretive errors, anatomic defects, imaging failure and inevitable errors. The first three of these error sources can be classified as “preventable” errors and the last two as non-preventable errors. False instrumentation may include problems caused by inadequate contact of balloon to irregular tumor surface and a gap formed by air, fluid or feces. Any images in this category would not give the examiner a clear and accurate view of the tumor, thereby reducing the likelihood of a correct diagnosis. The second preventable category, interpretive errors may be due to examiner confusion and lead mostly to the overstaging of a tumor even though the exam provided clear ultrasonic images. Interpretive errors would therefore be consistent with an examiner who, for example, had inadequate experience. Imaging error due to anatomic defects are not naturally occurring defects but those caused by previous biopsies, polypectomies, or operations resulting in edema, abscesses, or fibrosis. It is desirable to implement improvements in an ERUS system that would eliminate user dependent error.

Although ERUS is presently the standard of care for the accurate staging of rectal cancer, in its current format, it is limited by its 2D nature, lack of portability, and difficulty in obtaining accurate intra and inter examiner comparisons.

ERUS image interpretation is associated with a significant learning curve. One group characterized ERUS as a relatively simple procedure to learn, and once a moderate degree of experience is gained, should be routinely incorporated into the evaluation of rectal neoplasms. However, they also suggest that an examiner's interpretative skills would stay at a high level of accuracy after 15 ERUS exams have been performed. Yet another report defines a moderate number of exams as 30 per year, and if less than 30 are performed annually, it should be expected that the results would not be as accurate as possible. Therefore they conclude that the centralization of transrectal ultrasonography service is mandatory if a high level of quality is to be achieved with this method.

Inter-observer accuracy may be dependent upon stage. Several reports have demonstrated inter-observer agreement to be low for T2 tumors, high for T3 tumors, and that accurate interpretation of T1 and T2 tumors requires a very experienced clinician. This group and others have concluded that image interpretation explains some differences in reported accuracies among studies.

The accurate staging of rectal tumors is necessary because understaging may lead to undertreatment and overstaging results in potentially more invasive operations and subsequent increased morbidity and mortality. The current research describes the initial work to incorporate image guided therapy techniques to make this goal of a more accurate ERUS system feasible.

Once a rectal tumor has been accurately staged, the next decision is the appropriate treatment for the patient. This decision is very important because the extent and invasiveness of resection or other therapies used will determine local control, cure, need for adjuvant therapy, sphincter preservation, and preservation or loss of sexual and urinary functions. The number of potential treatments form a relatively short list, however, one of them has decisively been shown to be the optimal cure for most cases of rectal cancer. This option is surgery, which involves the resection of the cancerous tissue from the rectum. Other treatments include electrocoagulation for distal rectal cancer less than 4 cm in diameter (although only in debilitated patients), local intraoperative radiation therapy (IORT), chemotherapy, oral medication (aspirin, NSAIDS and COX-2 inhibitors), and immunotherapy. Of these, electrocoagulation and IORT are the only treatments that are currently being used completely independent of surgery and are intended only as palliation.

There are two broad classifications of operations, those with the intent to cure and preserve sphincter function and those that cure without preservation of sphincter function. The examples herein will focus on those operations with the intent to cure and will not take sphincter preservation into account. Currently there are different surgical procedures that are appropriate for different stages of tumors, as well as for tumors found in different parts of the rectum. However, there is generally agreement among surgeons that a 1 cm distal surgical margin and a tumor free circumferential margin are appropriate for most resections.

The first type of surgery is local excision with the intent to cure. There are four surgical approaches to local excision including transanal, trans-sacral, trans-sphincteric and transanal endoscopic microsurgery. Transanal excision is the most commonly performed. In this operation, the resected section is limited to the immediate area surrounding the tumor. This procedure is appropriate for tumors that are low stage, are less than about 3 cm in diameter, and are thought to be without lymph node metastases. Local excision can be completed with good margins and a low risk of recurrence for patients who are appropriately selected. With all of the benefit of local excision provides, it should again be noted that the correct staging of the tumor is significant in the success of the treatment.

Radical surgery has been shown to have the most impressive results is Total Mesorectal Excision (TME). The findamental principle of TME is a precise, careful anatomical dissection in the embryological plane that exists between the mesorectum, derived from the dorsal mesentery, and the parietal presacral fascia. The four main principles of TME include mesorectal dissection with direct visualization; Specimen-orientated surgery, the objective of which is an intact mesorectum with no tearing of the surface and no circumferential margin involvement; recognition and preservation of the autonomic nerves on which sexual and bladder function depend; and a major increase in sphincter preservation and reduction in the number of permanent colostomies. TME does require more time in operating room than other more traditional radical surgeries, however, it is currently thought to be the best option when radical excision is needed. TME's advantage is the low local recurrence without additional therapy.

One of the most fundamental forces in the development of surgery and other forms of directed therapy is the need to increase the information available to physicians and to place that information in both spatial and temporal contexts. The field of interactive image guided procedures, as it is known today, began in the 1980's and focused on tracking the surgical position in the physical space and display position in image space. This technique was first used in the field of neurosurgery and eventually crossed over into many other medical fields. IGPs have four basic components including image acquisition, image-to-physical-space registration, three-dimensional tracking, and display of imaging data and location. There has been much concurrent advancement in these four areas, which is a necessity for the timely incorporation of IGP into common medical practice.

Current research is showing the potential widespread use of three dimensional (3D) ultrasound within the medical community. Limitations of two dimensional (2D) ultrasound that are addressed by 3D imaging include: mentally transforming multiple 2D images to form a 3D impression of the anatomy and pathology is not only time-consuming and inefficient, but is also more importantly, variable and subjective, which can lead to incorrect decisions in diagnosis, and in the planning and delivery of therapy; diagnostic (e.g. obstetric) and therapeutic (e.g. staging and planning) decisions often require accurate estimation of organ or tumor volume; conventional 2D ultrasound techniques calculate volume from simple measurements of height, width and length in two orthogonal views, by assuming an idealized (e.g. ellipsoidal) shape, which can potentially lead to low accuracy, high variability and operator dependency; conventional 2D ultrasound is suboptimal for monitoring therapeutic procedures, or for performing quantitative prospective or follow-up studies, due to the difficulty in adjusting the transducer position so that the 2D image plane is at the same anatomical site and in the same orientation as in the previous examination; the validity of diagnostics with 3D as compared to 2D ultrasound is being tested, and the reported conclusions to date show 3D to be superior.

Some reported techniques to collect 3D data from 2D ERUS probes include magnetic tracking, the timed pull-out method, and a rotating stepper motor for a stationary, side firing probe. For a 360-degree rotating ERUS probe, only optical tracking, magnetic tracking and the timed pullout method are viable options. Magnetic localization systems are currently not as accurate as optical localization systems.

The timed pullout method is also known as linear scanning. The timed pullout method can provide relatively accurate results, but requires a bulky device holding the ERUS probe and placed close to the patient. This device contains the stepper motor and controls the linear movement of the probe. Because of the size of the mechanical mechanism that must be used for the timed pullout method, and the fact that any accidental movement of the device would cause invalid data, optical ERUS tracking is more useful and less of a hindrance to the clinician in the procedural room.

It is desirable to provide an apparatus and method for calibration, tracking and volume construction data for use in image-guided procedures. Further, is desirable to provide an apparatus and method for calibration, tracking and volume construction data for use in image-guided procedures within the context of endorectal imaging and for the detection and staging of tumors.

### BRIEF SUMMARY OF THE INVENTION

Briefly stated, the present invention comprises an apparatus and method for calibration, tracking and volume construction data for use in image-guided procedures.

In one embodiment, present invention comprises an apparatus that collects and processes physical space data while performing an image-guided procedure on an anatomical area of interest. The apparatus includes a calibration probe that collects physical space data by probing a plurality of physical points, a tracked ultrasonic probe, a tracking device that tracks the ultrasonic probe in space and an image data processor comprising a computer-readable medium. The tracked ultrasonic probe outputs one of two-dimensional (2D) ultrasonic image data, three-dimensional (3D) ultrasonic image data and four-dimensional (4D) ultrasonic image data. The physical space data provides three-dimensional (3D) coordinates for each of the physical points. The computer-readable medium holds computer-executable instructions that includes determining registrations used to indicate position in both image space and physical space based on the physical space data collected by the calibration probe; using the registrations to map into image space, image data describing the physical space of the tracked ultrasonic probe used to perform the image-guided procedure and the anatomical area of interest; and constructing a three-dimensional volume based on the ultrasonic image data on a periodic basis.

In another embodiment, the present invention comprises a method of collecting and processing physical space data while performing an image-guided procedure on an anatomical area of interest. The method includes collecting physical space data by probing a plurality of physical points using a calibration probe. The physical space data provides three-dimensional (3D) coordinates for each of the physical points. An ultrasonic probe that outputs one of two-dimensional (2D) ultrasonic image data, three-dimensional (3D) ultrasonic image data and four-dimensional (4D) ultrasonic image data is tracked using a tracking device that tracks the ultrasonic probe in space. In an image data processor comprising a computer-readable medium holding computer-executable instructions (i) based on the physical space data collected by the calibration probe, determining registrations used to indicate position in both image space and physical space; (ii) using the registrations to map into image space, image data describing the physical space of the tracked ultrasonic probe used to perform the image-guided procedure and the anatomical area of interest; and (iii) constructing a 3D volume based on the ultrasonic image data on a periodic basis.

In another embodiment, the present invention comprises an article of manufacture for collecting and processing physical space data while performing an image-guided procedure on an anatomical area of interest. The article of manufacture includes a computer-readable medium holding computer-executable instructions for performing a method. The method includes collecting physical space data from a calibration probe that is used to probe a plurality of physical points. The physical space data provides three-dimensional (3D) coordinates for each of the physical points. Tracking data about an ultrasonic probe that outputs one of two-dimensional (2D) ultrasonic image data, three-dimensional (3D) ultrasonic image data and four-dimensional (4D) ultrasonic image data is received from a tracking device that tracks the ultrasonic probe in space. Based on the physical space data collected by the calibration probe, registrations used to indicate position in both image space and physical space are determined. The registrations are used to map into image space, image data describing the physical space of the tracked ultrasonic probe used to perform the image-guided procedure and the anatomical area of interest. A 3D volume is constructed based on the ultrasonic image data on a periodic basis.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown. In the drawings:

FIG. 1 shows a hardware system for one possible configuration of an image-guided procedure tracking system in accordance with preferred embodiments of the present invention;

FIG. 2 is a cross-sectional representation of a rectum including muscular and mucosal layers;

FIG. 3 is an ultrasonic image of a rectal wall acquired by an endorectal ultrasonic (ERUS) probe;

FIG. 4 is a diagram of a rectal wall including muscular and mucosal layers and showing various stage tumors (TX, T0, T1, T2, T3 and T4);

FIG. 5 is an ultrasonic image of a rectal wall having a tumor acquired by an endorectal ultrasonic (ERUS) probe;

FIG. 6 is a perspective view of a tracked endorectal ultrasonic (TERUS) probe for use with preferred embodiments of the present invention;

FIG. 7 is a side elevational view of a rectal phantom (experimental set-up) having silicon layers and being inserted into a polyvinylchloride (PVC) pipe with fiducial markers disposed thereon;

FIG. 8 is an ultrasonic image of a rectal phantom having a simulated tumor acquired by a tracked endorectal ultrasonic (TERUS) probe in accordance with the preferred embodiments of the present invention;

FIG. 9 is a computed tomography (CT) image of a rectal phantom having a simulated tumor corresponding to the ultrasonic image of FIG. 8;

FIG. 10 is a perspective view of the tracked endorectal ultrasonic (TERUS) probe of FIG. 6 being immersed in a water-bath for calibration;

FIG. 11 is a perspective view of a calibration probe and a reference emitter for an optically tracked system, each having a plurality of infrared emitting diodes (IREDs) disposed thereon for use with the image-guided procedure tracking system of FIG. 1;

FIG. 12 is a perspective view of an image-guided procedure tracking system in accordance with preferred embodiments of the present invention;

FIG. 13 is an ultrasonic image showing a tip of a calibration probe acquired by a tracked endorectal ultrasonic (TERUS) probe during a calibration procedure in accordance with the preferred embodiments of the present invention;

FIG. 14 is a plot of collected calibration data points (*) and a calculated plane (+) acquired by an image-guided procedure tracking system in accordance with preferred embodiments of the present invention;

FIG. 15 a computed tomography (CT) image of a rectal phantom having a fiducial marker affixed thereto;

FIG. 16 a perspective view of a fiducial marker for use with the image-guided procedure tracking system of FIG. 1;

FIG. 17 shows a hardware system for one possible configuration of an optical tracking system in accordance with preferred embodiments of the present invention;

FIG. 18 shows a general flow chart for an image-guided tracking system in accordance with preferred embodiments of the present invention;

FIG. 19 shows a basic software architecture for one possible configuration of an image-guided tracking system in accordance with preferred embodiments of the present invention; and

FIG. 20 shows a general flow chart for an image-guided tracking system for one possible configuration of an image-guided tracking system in accordance with preferred embodiments of the present invention.

### DETAILED DESCRIPTION OF THE INVENTION

Certain terminology is used in the following description for convenience only and is not limiting. The words “right”, “left”, “lower”, and “upper” designate directions in the drawings to which reference is made. The words “inwardly” and “outwardly” refer direction toward and away from, respectively, the geometric center of the object discussed and designated parts thereof. The terminology includes the words above specifically mentioned, derivatives thereof and words of similar import. Additionally, the word “a”, as used in the claims and in the corresponding portions of the specification, means “one” or “at least one.”

Preferred embodiments of the present invention include Image Guided Procedures (IGP). IGP have four basic components: image acquisition, image-to-physical-space registration, three-dimensional tracking, and display of imaging data and location. A relevant IGP system is disclosed in U.S. Pat. No. 6,584,339 B2 (Galloway, Jr. et al.), the contents of which is incorporated by reference herein. U.S. Pat. No. 6,584,339 B2 is attached hereto as an Appendix. In IGP, physical space data provides three-dimensional (3D) coordinates for each of the physical surface points. Based on the physical space data collected, point-based registrations used to indicate position in both image space and physical space are determined. The registrations are used to map into image space, image data describing the physical space of an instrument used to perform the IGP, an anatomical region of interest and a particular portion to be studied (e.g., a tumor or growth). The image data is updated on a periodic basis.

Further, preferred embodiments of the present invention utilize an optically tracked two dimensional (2D) ultrasound probe to acquire a 3D image(s). Embodiments of the present invention, therefore permit the creation of a 3D ultrasound volume from 2D tracked ultrasound data. Acquired imaging scans (e.g., Computed Tomography (CT) scans) can be used as a comparison and/or in conjunction with pre-operative and inter-operative 3D ultrasound volume sets. The 3D ultrasound volume sets provide the ability to be tracked over time. Optionally, the ultrasound probe outputs one of two-dimensional (2D) ultrasonic image data, three-dimensional (3D) ultrasonic image data and four-dimensional (4D) ultrasonic image data.

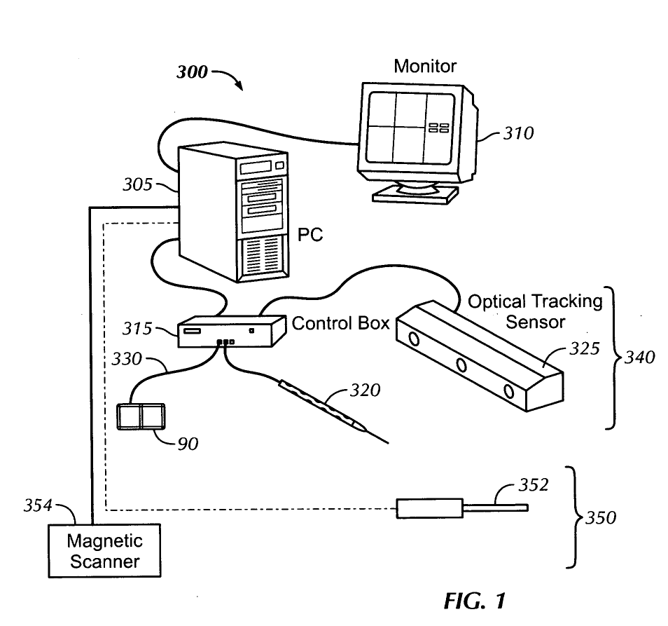

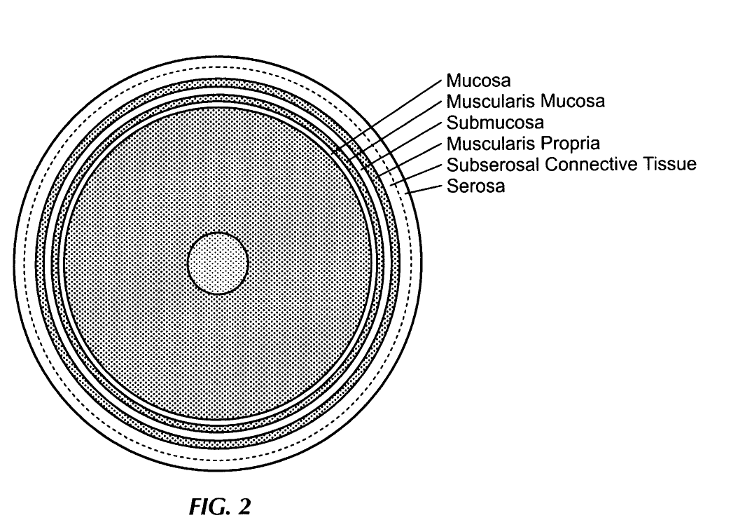

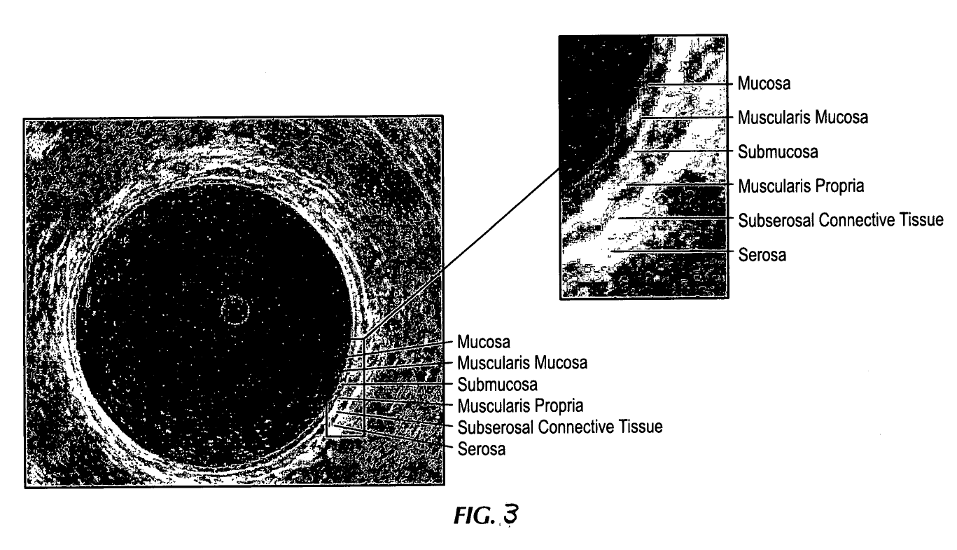

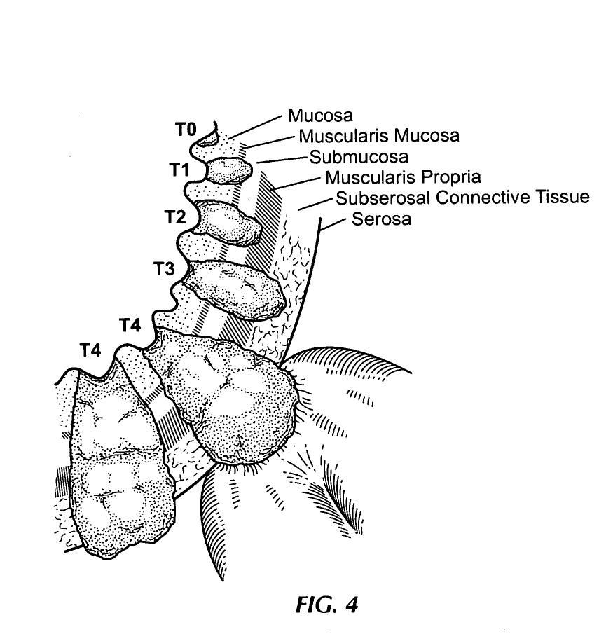

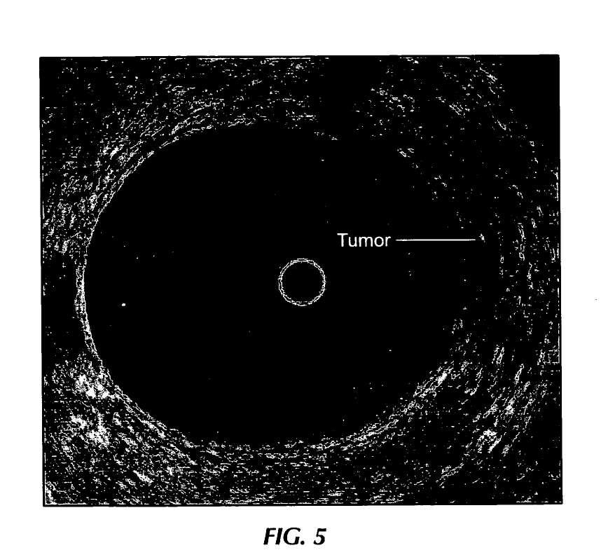

Besides ease of use, optical tracking has an advantage over the other 3D acquisition methods which is a result of coordinate integrated imaging. When using a tracked ultrasonic device, such as a tracked ultrasonic probe 352(FIG. 1), the exact location and orientation of the probe are known. Through the process of calibration, the location and orientation of the ultrasound beam are also known in an external coordinate space. This allows each pixel in the ultrasound data set to be assigned a 3D coordinate value in a physical space that is related to the ultrasound space through a specific transformation matrix. This method of assigning coordinate values to the ultrasound data in physical space has two advantages. First it allows the direct comparison of two imaging modalities. This is achieved by transforming a data set, such as CT, into the same physical space as the ultrasound making an accurate comparison possible. Second this method allows localization of multiple tools and image sets into the same physical space. The user then has the ability to guide a tracked instrument, such as a biopsy needle or surgical instrument, to a specific location in physical space while at the same time viewing the progress in all imaging modalities (i.e., ultrasound and CT). These co-registration and guidance techniques are not possible using the mechanical 3D volume reconstruction methods because multiple image sets and surgical tools cannot be localized in the same physical space. The mechanical based methods are appropriate for 3D volume reconstruction of ultrasound data, but are not valid for anything beyond visual enhancement of the rectum.

Referring to FIG. 1, an apparatus 300 that collects and processes physical space data while performing an image-guided procedure on an anatomical area of interest includes a calibration probe 320 that collects physical space data by probing a plurality of physical points, a tracked ultrasonic probe 352that outputs two-dimensional ultrasonic image data, a tracking device 325 that tracks the ultrasonic probe 352in space and an image data processor 305 comprising a computer-readable medium (e.g., memory, FlashRAM, hard disk, etc.). The tracked ultrasonic probe 352 may output any one of two-dimensional (2D) ultrasonic image data, three-dimensional (3D) ultrasonic image data and four-dimensional (4D) ultrasonic image data. The physical space data provides 3D coordinates for each of the physical points. The computer-readable medium holds computer-executable instructions that include determining registrations used to indicate position in both image space and physical space based on the physical space data collected by the calibration probe 320; using the registrations to map into image space, image data describing the physical space of the tracked ultrasonic probe 352 used to perform the image-guided procedure and the anatomical area of interest; and constructing a 3D volume based on the 2D, 3D, or 4D ultrasonic image data on a periodic basis.

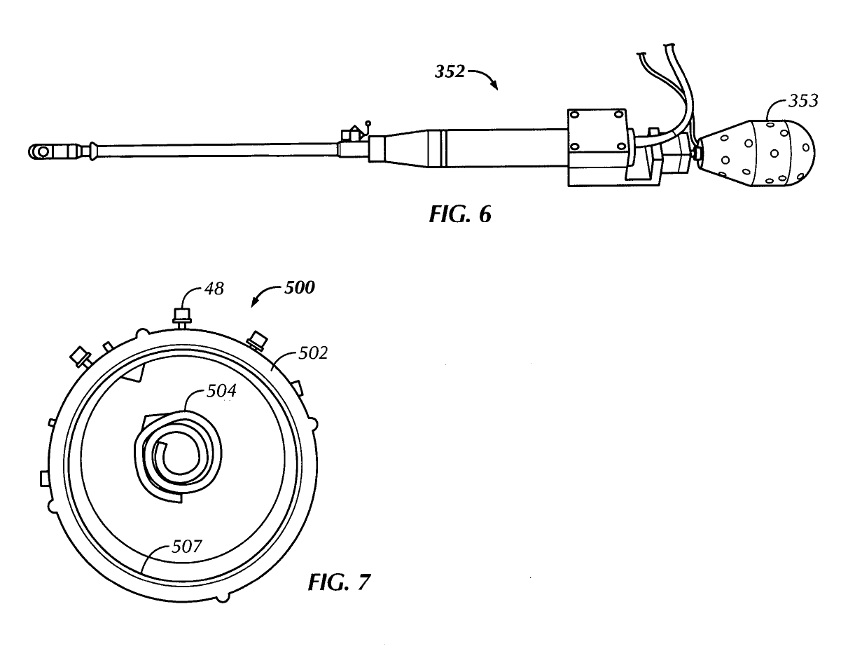

In one preferred embodiment of the present invention described using FIG. 1, an optically tracked endorectal ultrasound (TERUS) probe 352is utilized for improving the care of rectal cancer. It is desirable to provide a more accurate Endorectal Ultrasound (ERUS) system, and the incorporation of image guidance makes this goal feasible. ERUS images are intrinsically different from images taken by CT or Magnetic Resonance Imaging (MRI) in that ultrasound typically provides 2D images while CT and MRI provide 3D data sets that can be viewed as 2D images. By optically tracking ERUS one may overcome the limitations of 2D ultrasound and improve the diagnosis and care of patients with rectal cancer. The ERUS system may utilize a 360-degree rotating BK 1850 TERUS probe 352 (FIG. 6) commercially available from B-K Medical, Herlev, Denmark.

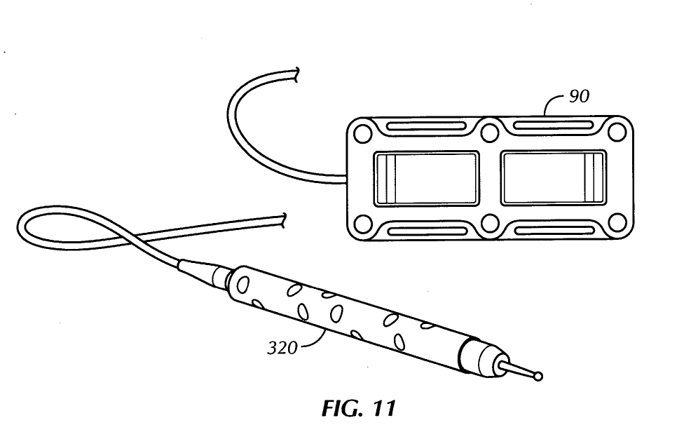

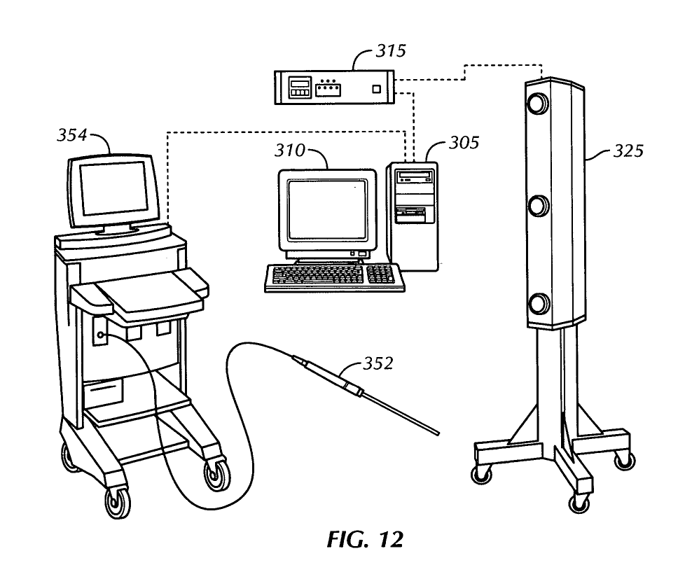

FIG. 1 shows that the ultrasound-based IGP system 300 includes an endorectal ultrasound probe 352 with an attached “gear shift knob” rigid body 353 (shown in FIG. 6), an ultrasound machine 354, a reference emitter 90, a calibration probe 320 and an optical tracking localization system 340. The reference emitter 90 establishes an overall 3D coordinate system. The ultrasound machine 354 may be a BK Falcon 2101 commercially available from B-K Medical (FIG. 12). The calibration probe 320 (FIG. 11) may be a pen probe commercially available from Northern Digital, Waterloo, Ontario, Canada. The optical tracking localization system 340 may be an Optotrak 3020 commercially available from Northern Digital.

The optical tracking system 340 determines triangulated position data based on emissions from a plurality of infrared emitting diodes (IREDs) distributed over the surface of a handle of the calibration probe 320, the TERUS probe 352 and/or another instrument. The optical tracking system 340 includes the optical tracking sensor 325 and optionally an optical reference emitter 90. The optical tracking sensor tracks the IREDS that are disposed on the handle of the calibration probe 320 and IREDS disposed on the reference emitter 90. The reference emitter 90 is rigidly or semi-rigidly attached to the patient. FIGS. 1 and 11 show a rectangularly shaped reference emitter 90, but other shaped reference emitters 90 may be utilized such as a cross-shape reference emitter 90′ (FIG. 17) and the like. The plurality of IREDs emit a plurality of intermittent infrared signals used to triangulate the position of the calibration probe 320 in 3-D image space. By using the point-based registrations and the triangulated position data to map into image space, image data describing the physical space of the distal end of the TERUS probe 352 can also be used to perform the IGP and to update the image data on a periodic basis. Other image-tracking systems such as binocular-camera systems may be utilized without departing from the present invention.

FIGS. 1 and 17 show that the optical tracking localization system 340 includes a control box 315 to interface with a computer 305. The software program that allows the integration of the components may be an operating room image-oriented navigation system (ORION), which was developed in the Surgical Navigation and Research Laboratory (SNARL) lab at Vanderbilt University, Nashville, Tenn. ORION may be implemented in Windows NT using MS Visual C++6.0 with the Win32 API. ORION was originally developed in Windows NT and is running on a 400 MHz processor personal computer (i.e., an image data processor) 305 with 256 MB of memory and a display monitor 310. However, other operating systems and processors may be utilized. The computer 305 may also include two specialized cards such as a VigraVision-PCI card (commercially available from VisiCom Inc., Burlington, Vt.) which is a combination color frame grabber and accelerated SVGA display controller which is capable of displaying NTSC video images in real time, and an ISA high-speed serial port card communicates with the calibration probe 320 via the control box 315. Of course, the computer 305 may include the necessary interfaces and graphics drivers without the need from specialized cards. For example, optical tracking system 340 may include network connections such as Ethernet, infrared (IR), wireless (Wi-Fi), or may include bus adapters such as parallel, serial, universal serial bus (USB) and the like.

Alternatively, the tracking system 340 may include a magnetic tracking device in a handle of the tracked ultrasonic probe 352 that is tracked by magnetic tracking techniques.

Alternatively, the tracking system 340 may include radiofrequency (RF) tracking using an RF signal generator that is built into a handle of the tracked ultrasonic probe 352.

Alternatively, the tracking system 340 may include gyroscopic tracking of the tracked ultrasonic probe 352.

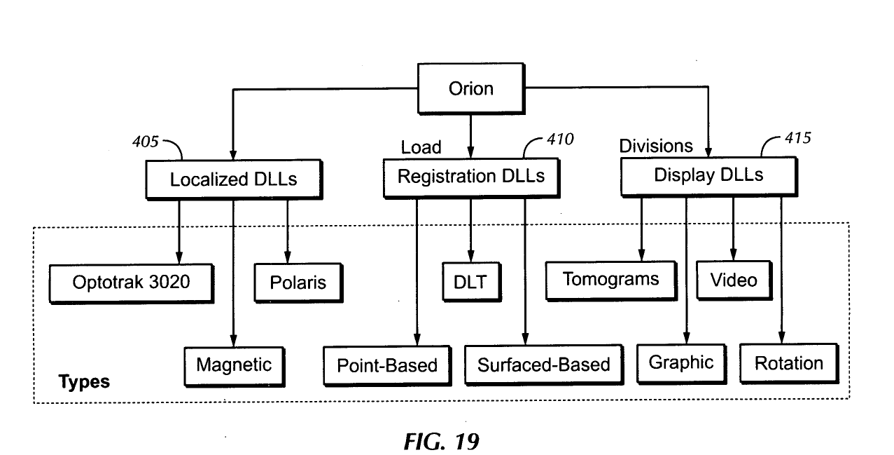

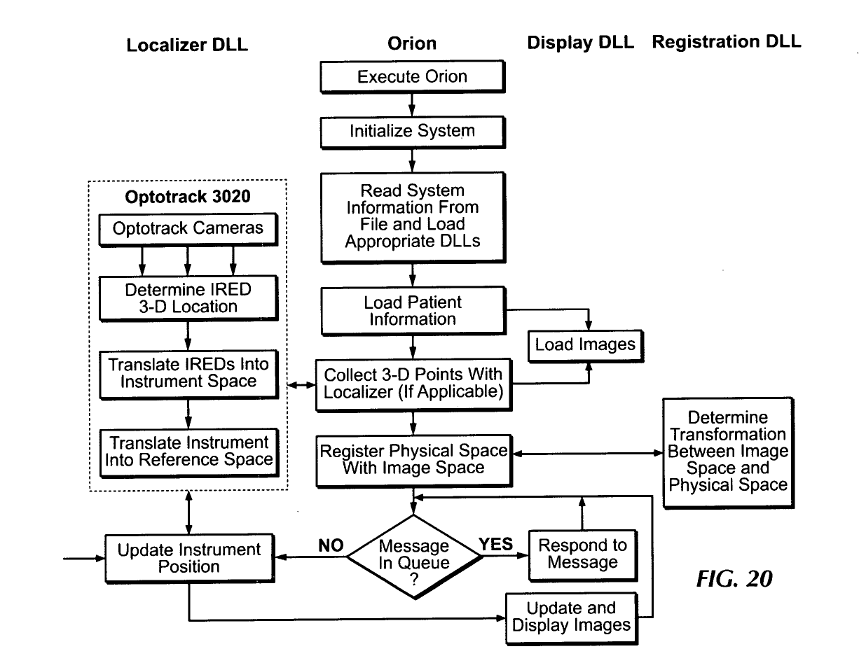

FIG. 19 shows a basic software architecture for one possible configuration of an image-guided tracking system 340 in accordance with preferred embodiments of the present invention. The software may include dynamic link libraries (DLLs) such as localizer DLLs 405, registration DLLs 410 and display DLLs 415. FIG. 20 shows a general flow chart for an image-guided tracking system for one possible configuration of an image-guided tracking system in accordance with preferred embodiments of the present invention.

Other hardware, operating systems, software packages, and image tracking systems may utilized without departing from the present invention.

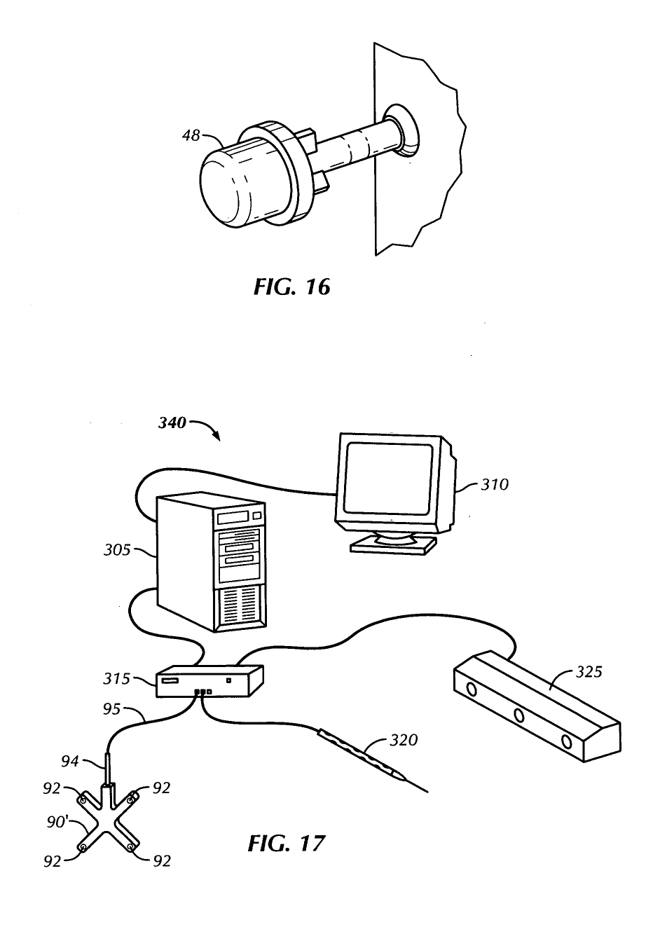

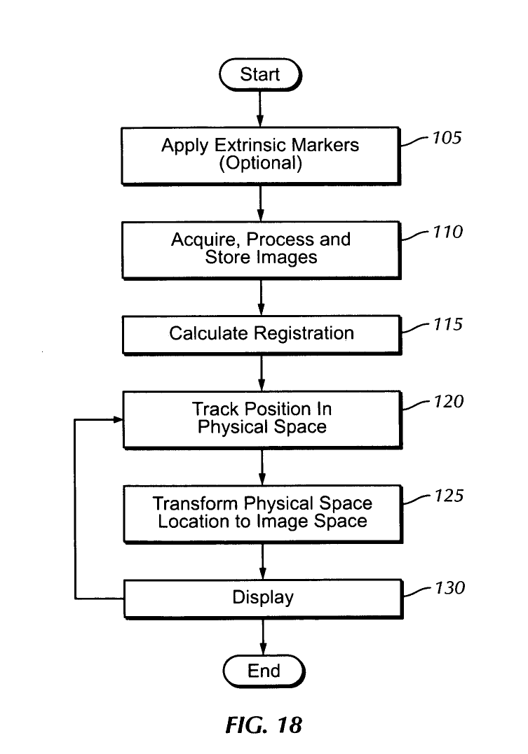

Locations of targets are found using two different imaging modalities, CT and ultrasound, and the target registration error (TRE) between these two image sets are calculated. Fiducial markers 48 (FIG. 16) are used for image registration. The fiducial markers 48 may either be skin markers such as those commercially available from Medtronics, Inc., Minneapolis, Minn., or bone implant markers such as those commercially available from ZKAT, Hollywood, Fla. The fiducial markers 48 are utilized to localize in the images using image processing routines and then touch using an optical tracker in the operating room. The positions of the fiducials are recorded and then a point registration is performed using either a quaternion based or singular-value-decomposition-based algorithm. Fiducial divot caps are used for finding the location of the fiducials in physical space and fiducial CT caps are used for finding the location of the fiducials in CT space. These fiducial caps are interchangeable and an appropriate type was chosen depending on the desired imaging modality. Preferably, non-planar fiducials are used to align the CT and Ultrasound images in a rigid registration process.

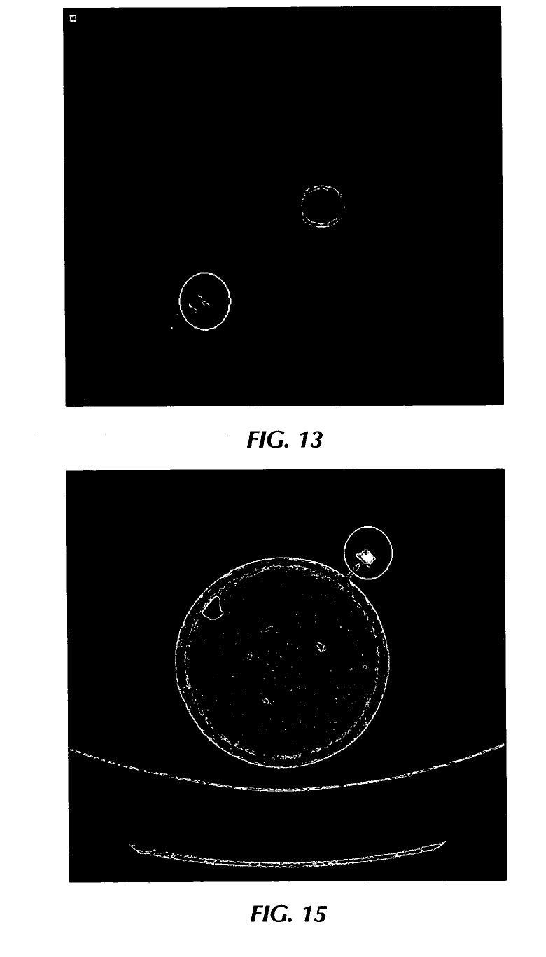

For calibration, two rigid bodies are used that both have Infrared Light Emitting Diodes (IREDS) and are tracked by the optical tracking localization system. One rigid body 353 was attached to the TERUS probe 352, and the TERUS probe 352 was securely fixed. The rotating tip of the TERUS probe 352is immersed in a bath of water or other suitable material. The ERUS mounted rigid body 352 functions as a reference to which the second rigid body is tracked. The second rigid body is the pen probe 320 with a ball-tip such as a 3 mm ball-tip. The tip of the calibration probe 320 is placed into the beam of the TERUS probe 352 and can be seen as a bright spot in a corresponding ultrasound image (see FIG. 13 with the location of the ball tip circled). Using the ORION-based system 300, which includes frame-grabbing capabilities, images along with the corresponding locations of the rigid bodies are acquired and saved. These images are processed to determine the “best-fit” plane through the points. The 2D location of the tip of the calibration pen probe 320 is determined in each of the images. A plurality of the 2D point locations are used to perform the calibration. The 2D locations are mapped to respective 3D locations using the Levenberg-Marquardt algorithm to solve for the resulting transformation matrix and the pixel to millimeter scale factors in the x and y directions. A subset of the acquired points or other acquired points can be used an internal check of the precision of the plane. The software program selects the points in a random order, so each time the program is run, there is a potential for a different solution because of different points used. The software program then reports the average and maximum errors as well as the standard deviations for the calibration points and the check points. The program also reports the scale factors that are used to map pixels to millimeters and the transformation matrix of the calibrated beam to the rigid body attached to the ERUS transducer. One important error to observe is the RMS error of the check points (TRE). This is a quantification of plane fit quality. However, it is important to note that with one set of calibration data points, it is possible to get a variation in the checkpoint TRE. This is because the data points used to calculate the plane and the data points used to check the accuracy of the plane change each time the calibration is run.

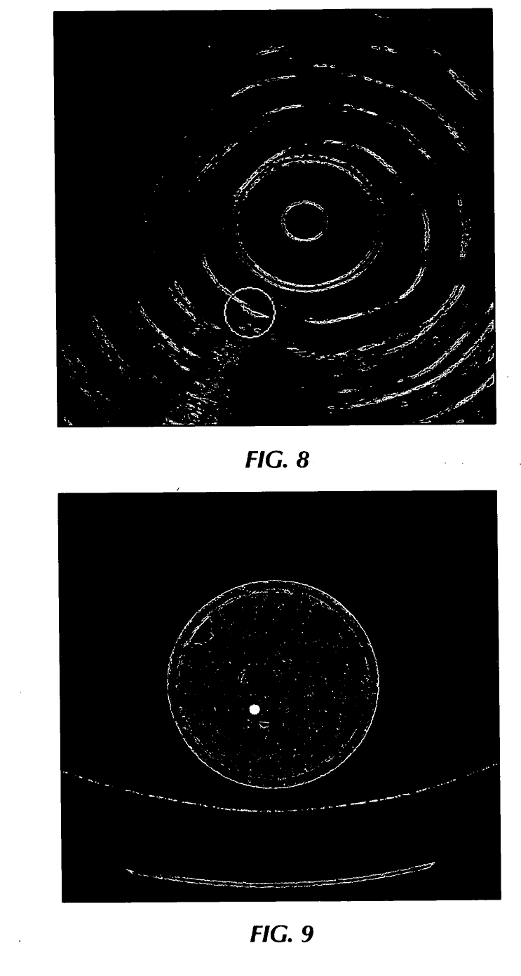

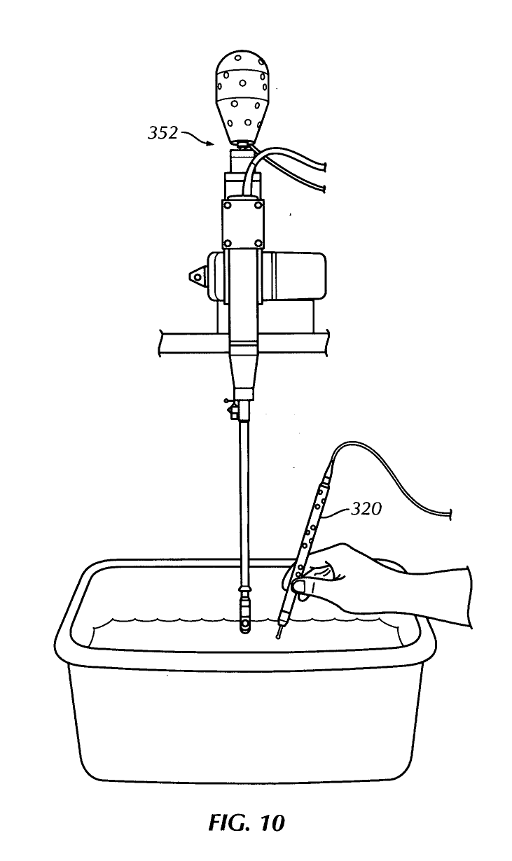

An experimental setup or “rectal phantom” 500 was created to simulate a human rectum and to test the TERUS-based IGP system 300. FIG. 7 is a side elevational view of the rectal phantom 500 (i.e., the experimental set-up) having silicon layers 504 and being inserted into a polyvinylchloride (PVC) pipe 507. Fiducial markers 48 are disposed on the PVC pipe 507 for calibration purposes. FIG. 8 is an ultrasonic image of the rectal phantom 500 having a simulated tumor (i.e., a titanium sphere) therein acquired by the TERUS probe 352. FIG. 9 is a CT image of the rectal phantom 500 having the simulated tumor therein corresponding to the ultrasonic image of FIG. 8.

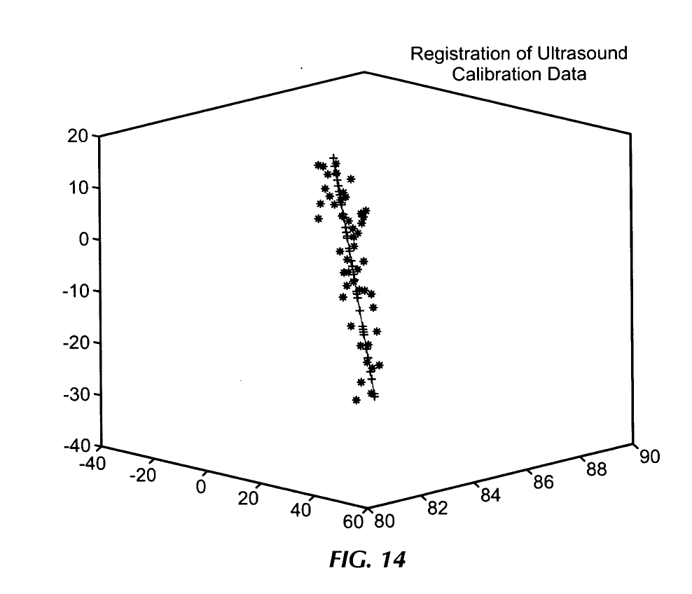

For exemplary purposes, FIG. 14 shows data points within the calculated plane (+) and the locations of the calibration data points (*) in a calibration experiment using 51 data points at 7.5 MHz. The error for the calibration used in these experiments is.presented in Table 2(a). A portion of the calibration error is actually caused by the beam thickness, and cannot accurately be defined by a single plane. However, the results using this calibration method have been shown to be adequate for rectal tumor localization.

TABLE 2(a)
ERUS Calibration Error (mm) at 10 MHz:
Calibration Error (mm) 10 MHz ERUS

Calib FRE = 1.1050
Avg Calib TRE = 1.4339

TABLE 2(b)
Rigid Registration Error (mm)
Registration Error (mm)

FRE = 0.5805
TRE = 0.72

TABLE 2(c)
Rectal Phantom Target Registration
Error (mm) at 10 MHz
Phantom TRE (mm)

TRE individual = 1.71 2.35 4.72 2.44 3.97
TRE average = 3.04

It is generally desirable to recalibrate the TERUS probe 352 when the rigid body 353 is removed and then reattached to the TERUS probe 352, and/or when the ultrasound is transmitted at a different frequency or viewed at a different field of view from the previous calibration. The second condition is of interest to particular TERUS probe 352 described above because this TERUS probe 352 includes a multi-frequency transducer that is easily interchangeable among the three frequencies and different fields of view.

The next step is the registration of the CT volume to the physical space. This is accomplished by using the extrinsic fiducials 48 that are attached to the outside of the patient's body and using rigid registration techniques. The (x,y,z) locations of the fiducials 48 are found in the CT set and in physical space, and then registered using the quaternion method. To find the locations in CT space, a three-dimensional volume is created from the tomogram set. The fiducial caps 48 used in the scan are radio-opaque and show up bright in the images (see e.g., FIG. 15).

The centroid of each fiducial cap 48 is found using an intensity based approach and interpolation within slices to provide an accuracy of about half of the slice thickness. This process is known as super-resolution. As previously described, the physical space locations are then determined using the tracked physical space calibration probe 320. The rigid registration is then performed with a subset of the fiducials 48 from the two data sets. This registration creates a transformation matrix that rotates and translates one set of data to match the orientation of the other set. This transformation matrix, along with the average error of all of the fiducials 48, are calculated. For example, FRE for the above experimental test is presented in Table 2(b). An accurate fiducial registration error is necessary for, but does not guarantee, an accurate target registration error. Therefore, one fiducial 48 or an additional fiducial 48 can be used as a target, like in the calibration process, to check the accuracy of the FRE. The RMS error of the additional fiducial 48 is reported as the registration TRE (see Table 2(b)).

The final stage in the process involved finding the (x,y,z) locations of the targets in the CT tomograms the same way that the fiducial locations were found. Then by using the output transformation matrix from the registration process, the locations of those points in physical space are calculated. The locations of the targets are also found using the TERUS probe 352, and their respective (x,y,z) locations in physical space are calculated using the transformation matrices from the calibration process and tracked image guided system. This provides two data sets containing the locations of the same targets in physical space located by two different methods. The values found using CT are taken to be the actual locations, and the values found using the TERUS probe 352 are compared to the actual. The distance between the locations of each target is then found, and is recorded as the target registration error (TRE). Exemplary individual TREs and the average TRE are reported in Table 2(c). It should be noted that an accurate TRE is the only true verification of an accurately tracked system.

The average TRE of the targets using the TERUS probe 352 is about 3.04 millimeters (mm). The required tumor margins for rectal cancer are on the order of centimeters (cm). Thus, use of a TERUS system 300 to improve ultrasound imaging in rectal cancer is possible. The incorporation of IGP techniques to pre-operative staging, inter-operative visualization and post-operative follow-up of rectal cancer with TERUS improves the accuracy of staging, reduces the overall morbidity and mortality rates and assists clinicians to detect and follow recurrences of rectal cancer over time.

By optically tracking the TERUS probe 352 as data is collected, the intensity value for each pixel can be saved and then inserted into the nearest voxel in a corresponding volume matrix. Then validation of the accuracy of a volume reconstruction can be performed by finding the 3D coordinates of targets that are inside of the volume and comparing them to known physical locations. Over-determining the data placed into the volume around the area of interest is desirable so that speckle can be reduced and the signal to noise in the area will be improved by averaging a particular pixel's value from different images. Transformation of a 2D coordinate location in ultrasound image space into its corresponding 3D physical space occurs pixel by pixel and any unfilled voxels are left empty. It is contemplated that vector mathematics can be utilized to speed up the process. During the transformation process, the intensity value of each pixel is placed into the appropriate 3D voxel. Multiple intensity values that map to the same voxel in 3D space are handled. Preferably, arithmetic averaging is used when multiple intensity values are mapped to the same voxel such that the average of the multiple intensity values is placed into the voxel. Alternately, when multiple intensity values are mapped to the same voxel in 3D space, the intensity value is overwritten into the voxel.

Of course, the preferred embodiments of the present invention are not limited to endorectal imaging and evaluation and may be utilized to analyze other anatomical regions of interest such as the vagina, the uterus, colon, stomach, upper intestine, pancreas, throat and the like. Tracking flexible ultrasound allows for applications in colon, esophageal, pancreatic, duodenal, and other such cancers. The use of rigid ultrasound probe in this invention allows for use in the liver, breast, kidney, prostate, and other such parts of the body that can be imaged by rigid ultrasound probe. Therefore, embodiments of the present invention include a tracked ultrasonic probe 352 that is one of a rigid body, a flexible body and a combination of a rigid body and a flexible body.

The National Cancer Institute reports the expected number of new cancer diagnosis for 2005 at http://seer.cancer.gov. The four largest areas of new cancer cases are (i) prostate, 232,090, (ii) breast, 211,240, (iii) colon and rectal, 145,290, and (iv) urinary/bladder, 63,210. The expected new cases of cancer of all types is to increase over time. The preferred embodiments may be utilized in conjunction with laparoscopic and endoscopic surgical techniques, as well, such as by inserting an ultrasonic probe 352into a body through a cannula.

Esophageal and pancreatic cancers remains quite lethal with low survival even in patients undergoing surgery. Endoscopic ultrasound is considered part of the standard for staging pre-operatively. The addition of tracked technologies may enable further treatment or selection criteria for patients with these diseases.

Prostate cancer is treated in a multitude of methods, surgically, external beam, and brachytherapy with radiation seeds as well as hormonal therapy. All modes of treatment rely on diagnostic ultrasound to determine the optimum method. Tracked devices may change the algorithms for treatment of this very common cancer.

Breast cancer can be caught early with screening however mammography has limitations of radiation as well as discomfort and post surgical changes. Tracked ultrasound has the potential to perform inter-exam comparisons in a digital manner and detect earlier lesions.

It is also contemplated that the tracked ultrasound probe 352 is one of a tracked angio-access ultrasound probe and a tracked intra-vascular ultrasound probe for use in arterial/vascular procedures like angiograms, by-passes, valve replacements or the like. It is further contemplated that the tracked ultrasound probe 352 is a tracked endoscopic ultrasound probe for gastrointestinal procedures. It is further contemplated that the tracked ultrasound probe 352 a tracked bronchoscope ultrasound probe for use in the lungs or airway. It is further contemplated that the tracked ultrasound probe 352 is a tracked arthroscopic ultrasound probe for use in joint procedures.

While described above as being used in combination with a CT scan, other imaging techniques such as Positron Emission Tomography (PET), Magnetic Resonance Imaging (MRI) and the like, may be utilized.

The ultrasound-based IGP system 300, when used with a second imaging technique (e.g., CT, PET, MRI and the like), enables other analysis such as size changes of a target (e.g., a tumor) in an anatomical region of interest (e.g., the rectum).

The present invention may be implemented with any combination of hardware and software. If implemented as a computer-implemented apparatus, the present invention is implemented using means for performing all of the steps and functions described above.

The present invention can be included in an article of manufacture (e.g., one or more computer program products) having, for instance, computer useable media. The media has embodied therein, for instance, computer readable program code means for providing and facilitating the mechanisms of the present invention. The article of manufacture can be included as part of a computer system or sold separately.

From the foregoing, it can be seen that the present invention comprises an apparatus and method for calibration, tracking and volume construction data for use in image-guided procedures, and more particularly, within the context of endorectal imaging and for the detection and staging of tumors. It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. Apparatus that collects and processes physical space data while performing an image-guided procedure on an anatomical area of interest, the apparatus comprising:
(a) a calibration probe that collects physical space data by probing a plurality of physical points, the physical space data providing three-dimensional (3D) coordinates for each of the physical points;
(b) a tracked ultrasonic probe that outputs one of two-dimensional (2D) ultrasonic image data, three-dimensional (3D) ultrasonic image data and four-dimensional (4D) ultrasonic image data;
(c) a tracking device that tracks the ultrasonic probe in space; and
(d) an image data processor comprising a computer-readable medium holding computer-executable instructions including:
(i) based on the physical space data collected by the calibration probe, determining registrations used to indicate position in both image space and physical space;
(ii) using the registrations to map into image space, image data describing the physical space of the tracked ultrasonic probe used to perform the image-guided procedure and the anatomical area of interest; and
(iii) constructing a 3D volume based on the ultrasonic image data on a periodic basis.

2. The apparatus according to claim 1, further comprising:
(e) a scanning device for scanning the respective anatomical area of interest of a patient to acquire, store and process a 3D reference of tissue, wherein the image data processor creates a scanned image based on the scanned tissue.

3. The apparatus according to claim 2, wherein the scanning device is one of a computerized tomography (CT) scanner, a magnetic resonance imaging (MRI) scanner and a positron emission tomography (PET) scanner.

4. The apparatus according to claim 2, wherein with functional cancer imaging similar to PET but specific for cancer or tissue markers.

5. The apparatus according to claim 1, wherein the tracking device includes an optical sensor and the tracked ultrasonic probe emits a plurality of intermittent infrared signals used to triangulate the position of the tracked ultrasonic probe instrument in 3D image space, the signals being emitted from a plurality of infrared emitting diodes (IREDs) distributed over the surface of a handle of the tracked ultrasonic probe.

6. The apparatus according to claim 1, wherein the tracked ultrasonic probe is one of a tracked endorectal ultrasound probe, a tracked transvaginal ultrasound probe and a tracked laparoscopic ultrasound probe.

7. The apparatus according to claim 1, wherein the tracking device includes passive tracking devices disposed on external portions the tracked ultrasonic probe.

8. The apparatus according to claim 1, wherein the tracking device includes a magnetic tracking device disposed in a handle of the tracked ultrasonic probe that is tracked by magnetic tracking.

9. The apparatus according to claim 1, wherein the tracking device includes radiofrequency (RF) tracking using an RF signal generator that is built into a handle of the tracked ultrasonic probe.

10. The apparatus according to claim 1, wherein the tracking device includes gyroscopic tracking.

11. The apparatus according to claim 1, wherein the tracked ultrasound probe is a tracked endoscopic ultrasound probe for gastrointestinal procedures.

12. The apparatus according to claim 1, wherein the tracked ultrasound device is a tracked bronchoscope ultrasound probe for use in the lungs or airway.

13. The apparatus according to claim 1, wherein the tracked ultrasound probe is one of a tracked angio-access ultrasound probe and a tracked intra-vascular ultrasound probe.

14. The apparatus according to claim 1, wherein the tracked ultrasound probe is a tracked arthroscopic ultrasound probe.

15. The apparatus according to claim 1, wherein the apparatus is calibrated to determine the location and orientation of the tracked ultrasonic probe in an external coordinate space and the location and orientation of an ultrasonic beam emitted by the tracked ultrasonic probe are determined in the external coordinal space by the image data processor.

16. The apparatus according to claim 1, wherein, when used with a second imaging technique, additional analysis is performed including one of detecting size changes of a target and detecting shape changes of the target.

17. The apparatus according to claim 1, wherein a plurality of instruments/tools are tracked by the tracking device and location data about each of the plurality of instruments/tools are registered into the 3D volume by the image data processor.

18. The apparatus according to claim 1, wherein 2D locations are mapped to respective 3D locations using the Levenberg-Marquardt algorithm to solve for a resulting transformation matrix and pixel to millimeter scale factors in the x and y directions.

19. The apparatus according to claim 1, wherein the tracked ultrasonic probe is one of a rigid body, a flexible body and a combination of a rigid body and a flexible body.

20. A method of collecting and processing physical space data while performing an image-guided procedure on an anatomical area of interest, the method comprising:
(a) collecting physical space data by probing a plurality of physical points using a calibration probe, the physical space data providing three-dimensional (3D) coordinates for each of the physical points;
(b) tracking an ultrasonic probe that outputs one of two-dimensional (2D) ultrasonic image data, three-dimensional (3D) ultrasonic image data and four-dimensional (4D) ultrasonic image data using a tracking device that tracks the ultrasonic probe in space; and
(c) in an image data processor comprising a computer-readable medium holding computer-executable instructions:
(i) based on the physical space data collected by the calibration probe, determining registrations used to indicate position in both image space and physical space;
(ii) using the registrations to map into image space, image data describing the physical space of the tracked ultrasonic probe used to perform the image-guided procedure and the anatomical area of interest; and
(iii) constructing a 3D volume based on the ultrasonic image data on a periodic basis.

21. An article of manufacture for collecting and processing physical space data while performing an image-guided procedure on an anatomical area of interest, the article of manufacture comprising a computer-readable medium holding computer-executable instructions for performing a method comprising:
(a) collecting physical space data from a calibration probe that is used to probe a plurality of physical points, the physical space data providing three-dimensional (3D) coordinates for each of the physical points;
(b) receiving tracking data about an ultrasonic probe that outputs one of two-dimensional (2D) ultrasonic image data, three-dimensional (3D) ultrasonic image data and four-dimensional (4D) ultrasonic image data from a tracking device that tracks the ultrasonic probe in space;
(c) based on the physical space data collected by the calibration probe, determining registrations used to indicate position in both image space and physical space;
(d) using the registrations to map into image space, image data describing the physical space of the tracked ultrasonic probe used to perform the image-guided procedure and the anatomical area of interest; and
(e) constructing a 3D volume based on the ultrasonic image data on a periodic basis.
